(19) United States
(12) Patent Application Publication
(10) Pub. No.: US 2005/0171057 A1
(43) Pub. Date: Aug. 4, 2005

(54) Pantoprazole cyclodextrin inclusion complexes

(75) Inventors: Ferdinando Giordano, Torre D'Isola Pvia (IT); Lucia Marzocchi, Pontremoli Massa Carrara (IT); Jose Ramon Moyano, Seville (ES); Alessandra Rossi, Stradella (IT)

Correspondence Address: NATH & ASSOCIATES PLLC, 1030 FIFTEENTH STREET, N.W., SIXTH FLOOR, WASHINGTON, DC, 20005, US

(73) Assignee: ALTANA PHARMA AG, KONSTANZ (DE)

(21) Appl. No.: 10/501,295

(22) PCT Filed: Jan. 13, 2003
(86) PCT No.: PCT/EP03/00242

(30) Foreign Application Priority Data
Jan. 15, 2002 (EP) 0200288.7
Mar. 22, 2002 (EP) 02006454.9

(51) Int. Cl. A61K 31/724; C08B 37/16

(52) U.S. Cl. 514/58; 514/338; 536/46

(57) ABSTRACT

An inclusion complex formed from pantoprazole and cyclodextrin is described.

### TECHNICAL FIELD

The present Invention relates to the field of pharmaceutical technology and describes pantoprazole cyclodextrin inclusion complexes.

### BACKGROUND ART

H⁺/K⁺-ATPase inhibitors, in particular pyridin-2-ylmethylsulfinyl-1H-benzimidazoles like those disclosed, for example, in EP-A-0 005 129, EP-A-0 166 287, EP-A-0 174 726 and EP-A-0 268 956 are important In the therapy of disorders originating from increased gastric acid secretion. Examples of active ingredients from this group which are commercially available are 5-methoxy-2-[(4-methoxy-3,5-dimethyl-2-pyridinyl)methylsulfinyl]-1H-benzimidazole (INN: omeprazole), 5-difluoromethoxy-2-[(3,4-dimethoxy-2-pyridinyl)methylsulfinyl]-1H-benzimidazole (INN: pantoprazole), 2-[3-methyl-4-(2,2,2-trifluoroethoxy)-2-pyridinyl)methylsulfinyl]-1H-benzimidazole (INN: lansoprazole) and 2-{[4-(3-methoxypropoxy)-3-methylpyridin-2-yl]methylsulfinyl}-1H-benzimidazole (INN: rabeprazole).

WO86/00913 discloses to form a stable complex by mixing and reacting omeprazole with β-cyclodextrin In 96% ethanol and cooling the reactant. WO93/13138 is related to a method for preparing enteric coated oral drugs containing acid-unstable compound, in particular an enteric-coated oral drug prepared in the form of acid-stable dosage unit inclusion complex formed by reacting benzimidazole derivative, acid-unstable compound, with cyclodextrin in alkaline solution. WO9638175 is related to a stabilized composition comprising an antiulcerative benzimidazole compound, particularly a proton pump inhibitor and a branched cyclodextrinic carboxylic acid. WO99/62958 is related to alkylated cyclodextrin derivates and their use as carriers for medicaments. WO98/40069 describes pharmaceutical formulations comprising a benzimidazole and as excipients, at least one cyclodextrin and at least one amino acid. EP-A-1018340 discloses the simultaneous reaction of a benzimidazole derivative with one or more amino acids and one or more cyclodextrins as a process to obtain an inclusion complex of a salt of a benzimidazole derivative.

### DESCRIPTION OF THE INVENTION

Surprisingly it has now been found that by reaction of pantoprazole with a cyclodextrin, inclusion complexes are obtained with increased overall solubility for pantoprazole brought about by the formation of soluble pantoprazole-cyclodextrin complexes.

Subject of the present invention is a pantoprazole cyclodextrin Inclusion complex.

Pantoprazole in connection with the invention refers to 5-difluoromethoxy-2-[(3,4-dimethoxy-2-pyridinyl)methylsulfinyl]-1H-benzimidazole. Pantoprazole is a chiral compound. The term pantoprazole in connection with the invention also encompasses the pure enantiomers of pantoprazole and their mixtures in any mixing ratio. Pure enantiomers which may be mentioned by way of example are (−)-pantoprazole and (+)-pantoprazole. Pantoprazole may be present as such or, preferably, in the form of its salts with bases. Examples of salts with bases which may be mentioned are sodium, potassium, magnesium or calcium salts. In a preferred embodiment pantoprazole refers to pantoprazole sodium or pantoprazole magnesium. If pantoprazole is isolated in crystalline form, it may contain variable amounts of solvent. The term pantoprazole therefore also represents according to the invention all solvates, in particular all hydrates, of pantoprazole and its salts.

In a preferred embodiment pantoprazole refers to pantoprazole sodium sesquihydrate (=pantoprazole sodium×1.5 H₂O), (−)-pantoprazole sodium sesquihydrate or pantoprazole magnesium dihydrate.

Cyclodextrin in connection with the invention preferably refers to α-, β- or γ-cyclodextrin, their hydrates, mixtures of α-, β- or γ-cyclodextrin or derivatives of α-, β- or γ-cyclodextrin such as alkyl or hydroxyalkyl derivatives. In a preferred embodiment of the invention cyclodextrin refers to β-cyclodextrin (β-CD), hydroxypropyl-β-cyclodextrin (HPβ-CD), the sodium salt of sulfobutylether β-cyclodextrin (SBβ-CD) or hydroxyethyl-β-cyclodextrin.

In a preferred embodiment according to the invention the pantoprazole cyclodextrin inclusion complex refers to a 1/1 (pantoprazole/cyclodextrin) complex. The pantoprazole cyclodextrin inclusion complexes may be produced for example by standard procedures for preparation of compound-cyclodextrin inclusion complexes. Such procedures are for example disclosed in WO86/00913, WO93/13138, WO96/38175 or by Duchene (in Proceedings of the Fourth International Symposium on Cyclodextrines, 265-275, 1988 by Kluwer Academic Publishers; eds. O. Huber and J. Szejtli). Inclusion compounds are usually prepared in liquid medium, but they can also be obtained in the solid phase. In one embodiment of the invention the inclusion complex is obtained by reacting pantoprazole with the cyclodextrin in a suitable solvent. In a preferred embodiment of the invention the solvent is an aqueous solvent or a solvent which essentially consists of an aliphatic alcohol, preferably ethanol. The inclusion complex may then be obtained by precipitation or freeze drying. In one embodiment the inclusion complex is obtained according to the method described in WO86/00913.

The pantoprazole cyclodextrin inclusion complexes of the invention can then be used as a basis for the production of the administration forms according to the invention. Administration forms according to the invention which may be mentioned, to which the preparations can be processed, are, for example, suspensions, gels, tablets, coated tablets, multicomponent tablets, effervescent tablets, rapidly disintegrating tablets, powders in sachets, sugar-coated tablets, capsules or alternatively suppositories. The excipients which are suitable for the desired administration forms are familiar to the person skilled in the art on the basis of his/her expert knowledge. Due to the increased solubility of pantoprazole In the pantoprazole cyclodextrin inclusion complex administration forms containing such inclusion complex have improved active compound bloavailability properties.

Suitable administration forms are for example disclosed in WO92/22284, WO97/02020, EP-A-0 244 380, WO96/01623, WO96/01624, WO96/01625 or WO97/25030.

The administration forms (also referred to as pharmaceutical formulations) according to the invention comprise the pantoprazole of pantoprazole cyclodextrin inclusion complex in the dose customary for the treatment of the particular disorder. The pantoprazole cyclodextrin inclusion complex of the invention can be employed for the treatment and prevention of all disorders which are regarded as treatable or preventable by the use of pyridin-2-ylmethylsulfinyl-1 H-benzimidazoles. In particular, the pantoprazole cyclodextrin inclusion complex of the invention can be employed for the treatment or prophylaxis of gastric disorders such as peptic ulcer disease or other disorders associated with gastric hyperacidity such as gastro-oesophagal reflux disease, Zollinger-Ellison Syndrome and dyspepsia (see e.g. MARTINDALE—The complete Drug Reference, Pantoprazole, MICROMEDEX® Healthcare Series Vol. 113, 2002). Administration forms such as tablets contain between 1 and 500 mg, preferably between 5 and 60 mg, of an acid-labile proton pump inhibitor. Examples which may be mentioned are tablets which contain 10, 20, 40 or 50 mg of pantoprazole. The daily dose (e.g. 40 mg of active ingredient) can be administered, for example, in the form of a single dose or by means of a plurality of doses of the tablets of the invention (e.g. 2×20 mg of active ingredient).

### EXAMPLE

### 5-difluoromethoxy-2-[(3,4-dimethoxy-2-pyridinyl)methylsulfinyl]-1H-benzimidazole Sodium Sesquihydrate β-cyclodextrin Inclusion Complex

1.73 g of 5-difluoromethoxy-2-[(3,4-dimethoxy-2-pyridinyl)methylsulfinyl]-1H-benzimidazole sodium sesquihydrate, 5.67 g β-cyclodextrin and 20 ml of ethanol (96%) are heated to 30 to 32° C. and stirred for 15 hours. The mixtures is cooled to 10° C. within 3 hours, filtered and the precipitate washed with ethanol (10° C.). After drying under reduced pressure the title compound is obtained.

### Complexation Studies of Pantoprazole with Cyclodextrins

Methods: Various solutions of the different cyclodextrins taken into consideration were prepared in phosphate buffer solution pH 7 at known concentrations and used to create saturated solutions of pantoprazole. After equilibration, these saturated solutions were filtered through MF™ membrane filters (pore size 0.45 μm), appropriately diluted with phosphate buffer solution and subjected to uv/vis spectrophotometric analysis. The cyclodextrin solutions prepared with phosphate buffer ranged from concentrations of 0% p/v up to 1.8% p/v for βCD, 20% p/v for HPβCD.

Results: In both cases, the solubility of pantoprazole increased markedly with increasing cyclodextrin concentration. In the presence of βCD pantoprazole's solubility rose to a four fold maximum with respect to its solubility in phosphate buffer whereas when equilibrated with HPβCD, pantoprazole showed an outstanding increase in its solubility reaching over seventy times that in phosphate buffer solution.

Conclusions: The phase solubility studies with βCD and HPβCD were both characterised by an over-all increase in pantoprazole's solubility brought about by the formation of soluble pantoprazole/cyclodextrin complexes. In the case of βCD phase solubility studies, the increase observed followed a typical Aₚ pattern commonly described by Higuchi-Connor, indicating the formation of complexes with an order higher than one. Pantoprazole's behaviour with HPβCD also followed a typical Higuchi-Connor's Aₚ pattern characterised further by an initially gradual increase in solubility.

Complexation studies on pantoprazole, both in its saline form as a sodium salt (PNTNa) and as a free acid (PNTH), were carried out with β-cyclodextrin (β-CD), hydroxypropyl-β-cyclodextrin (HPβ-CD) and the sodium salt of sulfobutylether β-cyclodextrin (SBβ-CD). A second study was carried to define the Phase-solubility behaviour of PNTH when equilibrated with aqueous solutions of β-CD, HPβ-CD and SBβ-CD prepared in phosphate buffer solution (pH 7).

### Methods of Sample Preparation

### Preparation of PNTH

Pantoprazole sodium salt was used as a starting material. PNTH was obtained via drop by drop acidification of an aqueous solution of the sodium salt with acetic acid (0.1 N) until pH 7.5 was reached. The resulting milky white suspension was filtered with use of the Buchner apparatus and the resulting solid was left to dry.

### Complexation Studies

Kneading

Inclusion complexes of pantoprazole, both the sodium salt (PNTNa) and the undissociated form (PNTH), with β-cyclodextrin (β-CD), hydroxypropyl-β-cyclodextrin (HPβ-CD) and the sodium salt of sulfobutylether β-cyclodextrin (SBβ-CD) were prepared via kneading 1:1 and 1:2 molar physical mixtures. A phosphate buffer or a 1:1 hydroethanolic solution were used as binding solutions respectively for the preparation of the inclusion complexes of PNNa and PNTH.

Freeze Drying

The technique of Freeze-Drying was applied to the preparation of 1:1 molar inclusion complexes of PNTH and PNTNa respectively with HPβ-CD.

In preparing the PNTNa: HPβ-CD 1:1 molar sample, which was then to be subjected to freeze drying, the exact quantity of HPβ-CD was first dissolved in the minimum amount of distilled water necessary (1.5 ml/mg ca) and stirred continuously. The exact amount of weighed PNTNa was then added in small portions. After complete solubilization of the drug, the sample was frozen in liquid nitrogen and freeze dried (−70° C., 760 torr) for 12 hours.

The PNTH: HPβ-CD 1:1 molar sample was prepared differently according to the following method. Once again, the exact quantity of HPβ-CD was first dissolved in the minimum amount of distilled water necessary (1.5 ml/mg ca) and stirred continuously. The exact amount of weighed PNTH was then added in small portions but its solubilization was helped along by the addition of acetone. The total volume of acetone added in relation to the amount of water used to solubilize HPβ-CD was 1:1. The sample was then frozen in liquid nitrogen and freeze dried (−70° C., 760 torr) for 12 hours.

### Phase-Solubility Studies of PNTH with Natural and Modified Cyclodextrins

The Solubility of PNTH in Phosphate Buffer Solution at pH 7 was Determined Using a Previously Constructed Asorbance vs. Concentration Calibration Curve.

A known amount of PNTH was suspended in cyclodextrin solutions of different concentrations ranging from; 0 to 1.8% (p/v) for β-CD, 0 to 20% (p/v) for H-Pβ-CD and 0 to 15% (p/v) for SB-β-CD. The resulting suspension was filtered using a PTFE 0.45μ filter, adequately diluted and then subjected to UV spectrophotometric analysis. The concentration of PNTH in the corresponding sample was then calculated referring to the relevant PNTH calibration curve.

### Characterization Techniques

Complexation Studies

Thermal traces were obtained using a Mettler DSC 821 ⁶ module. FTIR spectra were carried out on KBr discs in the 400-4000 cm⁻¹ range (Jasco FT-300-IE). The Freeze Dryer used was a Pirani 10 Edwards Modulyo model. Powder X-ray diffractograms were recorded using a Bruker D5005 Diffractometer.

Phase-Solubility Studies

UV-VIS spectra were recorded on a Jasco V570 Spectrophoto meter.

### Results

Phase-Solubility Studies

A. Phase-Solubility Curve of PNTH with β-CD in Phosphate Buffer Solution pH 7.

The Phase-solubility curve (FIG. 1) follows a typical Higuchi-Connor pattern of type A_{L}. In fact, pantoprazole presents a linear increase in its apparent solubility when equilibrated with solutions of β-CD, reaching up to four times its solubility in phosphate buffer alone (from 0.56 mmol/L up to 2.25 mmol/L).

[image]

B. Phase-Solubility Curve of PNTH with HPβ-CD In Phosphate Buffer Solution pH 7

Even in this case, the Phase Solubility curve (FIG. 2) follows a typically linear Higuchi-Connor pattern. In fact, the increase observed in the apparent solubility of pantoprazole when equilibrated with solutions of HPβ-CD, reaches up to 36 times that in phosphate buffer alone (from 0.56 mmol/L up to 17.8 mmol/L).

[image]

C. Phase-Solubility Curve of PNTH with SBβ-CD in Phosphate Buffer Solution pH 7

In this case, the solubility of pantoprazole increases linearly when equilibrated with solutions of SBβ-CD, and the Phase Solubility curve (FIG. 3) shows that its apparent solubility increases twenty fold with respect to its value in phosphate buffer alone (from 0.56 mmol/L up to 9.3 mmol/L).

[image]

Complexation Studies of Samples Prepared by Kneading

Differential Scanning Calorimetry (DSC) was first used to characterise both the physical mixtures and the solid phases obtained after wetting and kneading. In none of the cases did we succeed in obtaining an Inclusion complex using the technique of kneading. In fact, the DSC traces showed a first broad endothermic peak below 100° C. corresponding to cyclodextrin dehydration, and a second sharp endothermic peak either at 134° C. for PNTNa or at 144° C. for PNTH due to fusion.

Complexation Studies of Samples Prepared via Freeze Drying

The samples obtained via freeze drying both had a fluffy white foam-like consistancy. Thermal analysis of these samples demonstrated, by the absence of the characteristic endothermic peaks of fusion of PNTNa and PNTH, that complexation had effectively occurred. The PNTH-HPβ-CD 1:1 inclusion complex was extremely deliquescent. Powder X-ray diffractograms of the PNTNa: HPβ-CD 1:1 inclusion compound proved it to have an amorphous solid structure.

### CONCLUSIONS

1) Phase-solubility studies have shown that with all three CDs used, a notable increase in the apparent solubility of PNTH in phosphate buffer solution was observed.

2) Inclusion complexation was not achieved through kneading.

3) Freeze-drying permitted the preparation of an amorphous solid phase with HPβ-CD and PNTNa from their aqueous solution.

1. Inclusion complex formed of element A and element B, wherein element A is chosen from pantoprazole, a solvate of pantoprazole, a hydrate of pantoprazole, a salt of pantoprazole with a base, a solvate of a salt of pantoprazole with a base, a hydrate of a salt of pantoprazole with a base, an enantiomer of pantoprazole, a solvate of an enantiomer of pantoprazole, a hydrate of an enantiomer of pantoprazole, a salt of an enantiomer of pantoprazole, a solvate of a salt of an enantiomer of pantoprazole, a hydrate of a salt of an enantiomer of pantoprazole, or a mixture thereof; and wherein element B is chosen from cyclodextrin, a hydrate of cyclodextrin, a derivative of cyclodextrin or a mixture thereof.

2. Inclusion complex according to claim 1 wherein element A is chosen from pantoprazole sodium sesquihydrate (=pantoprazole sodium×1.5 H₂O), (−)-pantoprazole sodium sequihydrate, pantoprazole magnesium dihydrate or a mixture thereof.

3. Inclusion complex according to claim 1, wherein element B is β-cyclodextrin.

4. Method of preparing an inclusion complex according to claim 1 comprising reacting element A with element B in a suitable solvent.

5. Method according to claim 4, wherein the solvent is ethanol.

6. Inclusion complex prepared by the method according to claim 4.

7. Inclusion complex according to claim 1, which is a 1/1 inclusion complex.

8. Administration form comprising an inclusion complex according to claim 1 together with a suitable pharmaceutical auxiliary.

9. (canceled)

10. Method of treating or preventing a condition treatable or preventable with a pyridine-2ylmethylsulfinyl-1H-benzimidazole, which comprises administering to a subject prone to or afflicted with a condition a pharmaceutically acceptable administration form according to claim 8 comprising therapeutically effective amount of the inclusion complex.

11. Inclusion complex according to claim 2 wherein element B is β-cyclodextrin.

12. Method of preparing an inclusion complex according to claim 1 comprising reacting element A with element B in a suitable solvent.

13. Method of preparing an inclusion complex according to claim 3 comprising reacting element A with element B in a suitable solvent.

14. Administration form comprising an inclusion complex according to claim 2 together with a suitable pharmaceutical auxiliary.

15. Administration form comprising an inclusion complex according to claim 3 together with a suitable pharmaceutical auxiliary.
